(19) United States
(12) Patent Application Publication
(10) Pub. No.: US 2023/0126625 A1
(43) Pub. Date: Apr. 27, 2023

(54) Sustainable High Density Polyethylene and Process For Making Same

(71) Applicant: Celanese International Corporation, Irving, TX (US)

(72) Inventors: Rainer Walkenhorst, Melle (DE); Dominique Schroeer, Frankfurt Rhine-Main (DE); Britta Richter, Ingelheim (DE); Nina Nordlander, Hofheim (DE); Argiri Tsami-Schulte, Kelkheim (DE); Uwe Wertheimer, Aschaffenburg (DE); Jun Luo, Hicksville, NY (US)

(21) Appl. No.: 17/972,998

(22) Filed: Oct. 25, 2022

Related U.S. Application Data
(60) Provisional application No. 63/272,456, filed on Oct. 27, 2021.

(51) Int. Cl. C08F 10/02; B01J 20/26; A61L 27/16; A61L 27/56; B01D 71/26; H01M 50/417; H01M 50/431; H01M 50/449

(52) U.S. Cl. CPC C08F 10/02; B01J 20/267; A61L 27/16; A61L 27/56; B01D 71/261; H01M 50/417; H01M 50/431; H01M 50/449; C08F 2810/20

(57) ABSTRACT

High density polyethylene polymers, including high molecular weight and ultrahigh molecular weight polyethylene polymers, are disclosed that are at least partially made from bio-based feedstocks. The bio-based feedstocks are selected so as to produce high purity monomers capable of producing high density polymers for use in high purity applications, such as in producing implants and porous membranes for lithium-ion batteries.

### RELATED APPLICATIONS

The present application is based upon and claims priority to U.S. Provisional Patent Application Ser. No. 63/272,456, having a filing date of Oct. 27, 2021, and which is incorporated herein by reference.

### BACKGROUND

High density polyethylene polymers, and particularly high molecular weight polyethylene polymers and ultrahigh molecular weight polyethylene polymers or linear polyethylene polymers, are valuable engineering plastics with a unique combination of abrasion resistance, surface lubricity, chemical resistance, tensile strength, and impact strength. High density polyethylene polymers are used in numerous and diverse fields where the properties of the polymer can be tailored to the particular application.

For example, certain high density polyethylene particles having a high molecular weight can be sintered together and formed into various different filter devices. The filter devices can include filter funnels, immersion filters, filter crucibles, porous sheets, pen tips, marker nibs, aerators, diffusers, and lightweight molded parts.

High density polyethylene particles can also be combined with one or more plasticizers and gel extruded into films and fibers. For example, high density polyethylene polymers can be used to produce porous membranes. Porous membranes made from high molecular weight polyethylene polymers and ultrahigh molecular weight polyethylene polymers have significantly increased in importance and value due to the advent of the electric vehicle. For instance, the porous membranes can be used in lithium ion batteries as battery separators positioned between an anode and a cathode. Not only do membranes made from high density polyethylene polymers have optimum porosity characteristics, but also offer a shutdown temperature that provides safety to the battery into which the membrane is incorporated. In addition, conventional high molecular weight polyethylene polymers can be formed with very low impurities such that the polymer membrane does not in any way react with the chemical components contained in the battery.

High density polyethylene polymers are often used in conjunction with biomedical devices as well. High density polyethylene polymers, particularly ultrahigh molecular weight polyethylene polymers, for instance, have a purity sufficient for use in biological environments. For example, the polymers can be produced with a minimal concentration of residual catalyst and other impurities. As a result, high density polyethylene polymers can be used as load bearing components in prosthetic knee joints, prosthetic hip joints, and as bearing components for other prosthetic replacement joints for the human body.

High density polyethylene polymers are typically produced by polymerizing an ethylene monomer in the presence of a catalyst. In the past, ethylene monomers have been produced from crude oil through a catalytic cracking process. Over the years, the processes used to produce ethylene monomers have resulted in producing monomers with high purity that are well suited to producing higher molecular weight polyethylene, where exposure to the catalyst and reaction times are longer. Recently, however, many companies large and small have pledged to be carbon neutral within a particular time period. To be carbon neutral, a company must remove the same amount of carbon dioxide that it is emitting into the atmosphere to achieve a net-zero carbon emissions. A carbon negative company, on the other hand, removes more carbon from the atmosphere than it releases.

In view of the significant efforts across the globe of companies to go carbon neutral or to be carbon negative, a need exists for a process for producing high density polyethylene polymers in a more sustainable way without significantly changing the amount of impurities within the polymer or other characteristics of the polymer. A need also exists for polymer compositions and polymer products made from sustainable high density polyethylene polymers.

### SUMMARY

In general, the present disclosure is directed to producing high density polyethylene polymers, including high molecular weight polyethylene polymers and ultrahigh molecular weight polyethylene polymers, in a way that creates carbon offsets.

In one aspect, the present disclosure is directed to a polymer composition containing polymer particles comprising a high density polyethylene polymer. The high density polyethylene polymer can have a molecular weight of greater than about 300,000 g/mol and can have a density of greater than about 0.92 g/cm³. The high density polyethylene polymer has been formed from an ethylene monomer. In accordance with the present disclosure, at least a portion of the ethylene monomer comprises a bio-based ethylene that is made from one or more carbon negative or carbon neutral components. The bio-based content can be determined using a mass balance approach in one embodiment. Alternatively, the bio-based content can be determined according to ASTM Test D6866-21. The portion of the polymer made from carbon negative or carbon neutral components can be at least about 1%, such as at least about 10%. Alternatively, the bio-based content can be at least about 1%, such as at least about 10%, based on radiocarbon dating of Total Organic Carbon Content.

The high density polyethylene polymer, for instance, can be formed from a mixture of a fossil-based ethylene monomer and a bio-based ethylene monomer. The resulting high density polyethylene polymer can have a bio-based content or contain carbon negative or carbon neutral components in an amount of at least about 20%, such as at least about 30%, such as at least about 40%, such as at least about 50%, such as at least about 60%, and generally less than about 90%, such as less than about 80%, such as less than about 70%. In one embodiment, the high density polyethylene polymer can be formed exclusively from the bio-based ethylene monomer or can be formed exclusively from carbon negative or carbon neutral components.

The high density polyethylene polymer can have an average molecular weight of greater than about 500,000 g/mol, such as greater than about 700,000 g/mol, such as greater than about 1,000,000 g/mol, such as greater than about 1,300,000 g/mol, such as greater than about 1,700,000 g/mol, such as greater than about 2,000,000 g/mol, such as greater than about 2,500,000 g/mol, such as greater than about 3,000,000 g/mol, such as greater than about 3,500,000 g/mol, such as greater than about 4,000,000 g/mol, such as greater than about 4,500,000 g/mol, such as greater than about 5,000,000 g/mol, such as greater than about 5,500,000 g/mol, such as greater than about 6,000,000 g/mol, such as greater than about 6,500,000 g/mol, such as greater than about 7,000,000 g/mol, such as greater than about 7,500,000 g/mol, such as greater than about 8,000,000 g/mol, and less than about 12,000,000 g/mol. For purposes of the present specification, the molecular weights referenced herein are determined in accordance with the Margolies equation (“Margolies molecular weight”).

Ethylene monomers used to form the polyethylene polymer can originate from various different sources as long as the monomer remains high in purity and does not otherwise interfere with the ability of the monomer to be polymerized into a high density polyethylene including high molecular weight and ultrahigh molecular weight polyethylene polymers. The bio-based ethylene, for instance, can be formed from a carbon negative or carbon neutral component. In one aspect, the carbon negative or carbon neutral component comprises methane, such as derived from biomass. The methane can be subjected to a pyrolysis or a partial oxidation process for forming acetylene. The acetylene can then be hydrogenated into ethylene. Alternatively, the carbon negative or carbon neutral component can comprise ethanol that is converted into ethylene. The ethanol, for instance, can be a fermentation product. In still another embodiment, the carbon negative or carbon neutral component can comprise a vegetable oil or an animal fat. The vegetable oil or animal fat can be converted to ethylene by hydrodeoxygenation. In another embodiment, the carbon negative or carbon neutral component can comprise a tall oil which can be converted to ethylene.

The high density polyethylene polymer can be a Ziegler-Natta catalyzed polymer. The polymer particles can have an average particle size, D50, of from about 10 microns to about 1,000 microns, in one embodiment. The polymer particles can have a bulk density of from about 0.2 g/cm³ to about 0.54 g/cm³. The high density polyethylene polymer can have a melt flow rate of from about 0 g/10 min (not measurable) to about 20 g/10 min. The high density polyethylene polymer can be a polyethylene homopolymer or a polyethylene copolymer. For example, the polyethylene polymer can be a copolymer of ethylene and at least one comonomer comprising butene, propylene, hexene, or mixtures thereof. The butene, hexene, and/or the propylene can also be bio-based.

Various different articles can be made from the polymer composition. For instance, the polymer composition is well suited to producing medical implants. In one embodiment, the polymer is used to produce a battery separator comprising a porous membrane. The porous membrane can optionally include a coating on one surface of the membrane. The coating can comprise an inorganic coating or a polymer coating. The battery separator can be positioned within a battery between an anode and a cathode.

In still another embodiment, the polymer composition can be used to form a sintered article, such as a filter element.

Other features and aspects of the present disclosure are discussed in greater detail below.

### BRIEF DESCRIPTION OF THE DRAWINGS

A full and enabling disclosure of the present disclosure is set forth more particularly in the remainder of the specification, including reference to the accompanying figures, in which:

FIG. 1 is a cross-sectional view of a membrane for a battery made in accordance with the present disclosure.

Repeat use of reference characters in the present specification and drawings is intended to represent the same or analogous features or elements of the present invention.

### DETAILED DESCRIPTION

It is to be understood by one of ordinary skill in the art that the present discussion is a description of exemplary embodiments only and is not intended as limiting the broader aspects of the present disclosure.

In general, the present disclosure is directed to a process for producing high density polyethylene polymers in a more sustainable manner and to polymer compositions made from the high density polyethylene polymers. At least a portion of the feedstock that is used to produce the high density polyethylene polymers can be derived from biomass or other sustainable resources instead of being derived from fossil fuels, such as crude oil. The high density polyethylene polymers are produced from an ethylene monomer. In accordance with the present disclosure, the ethylene monomer can be derived from bio-based components, such as biogases, fermentation products, vegetable byproducts, animal byproducts, cellulosic byproducts, and the like. The bio-derived feedstock can be converted into ethylene and then used to produce the high density polymers which generally also have a high molecular weight. The high density polyethylene polymers produced according to the present disclosure have a much smaller carbon footprint and can even be produced so as to be overall carbon neutral or carbon negative.

High density polyethylene polymers, including high molecular weight and ultrahigh molecular weight polyethylene polymers, are commonly used in very specific applications where the purity of the polymer can be just as important as the mechanical properties. For example, high density polyethylene polymers used in biomedical applications should have ultrapure characteristics. Consequently, in the past, there has been a reluctance to change the monomer used to make the polymers, especially if the monomer is derived from other resources, such as byproducts. Of particular advantage, however, high density polyethylene polymers can be produced according to the present disclosure without sacrificing impurity levels or mechanical properties.

High density polyethylene polymers made according to the present disclosure can fulfill the sustainability needs of many manufacturers and consumers. The high density polyethylene polymers can be used to produce all different types of products and articles in all different fields. The high density polyethylene polymers, for instance, can be used to produce molded parts and articles for use in the medical field, automotive field, electrical field, the food handling industry, the water purification field, and the like. Manufacturers can incorporate the high density polyethylene polymers into their products in order to meet goals for renewable or bio-based content. Overall, the high density polyethylene polymers made according to the present disclosure can help manufacturers reduce their carbon footprint without in any way sacrificing quality or mechanical properties.

Ultimately, high density polyethylene polymers made according to the present disclosure can be certified according to any suitable standard. One such certification is the International Sustainability and Carbon Certification (ISCC). The ISCC is a globally applicable sustainability certification system and covers all sustainable feedstocks, including agricultural and forestry biomass, circular and bio-based materials and renewables. The ISCC follows the mass balance approach in which the renewable content of the polymer can be verified. In mass balance, renewable feedstock is attributed to selected products, according to their individual formulation taking into account all yields and losses. Only raw materials used as feedstock (but not for energy) for the production are considered for mass balancing. The key criteria used for applying the mass balance approach include feedstock qualification, chain of custody, and product claims.

The mass balance approach makes it possible to track the amount and sustainability characteristics of recycled and/or bio-based feedstocks in the value chain and attribute it to the final product in a verifiable manner. In one embodiment, the high density polyethylene of the present disclosure can be made exclusively from carbon negative or carbon neutral components under the mass balance approach. Alternatively, the high density polyethylene can be made from at least 20% carbon negative or carbon neutral components, such as at least about 30% carbon negative or carbon neutral components, such as at least about 40% carbon negative or carbon neutral components, such as at least about 50% carbon negative or carbon neutral components, such as at least about 60% carbon negative or carbon neutral components, such as at least about 70% carbon negative or carbon neutral components, such as at least about 80% carbon negative or carbon neutral components, and up to 100% carbon negative or carbon neutral components, such as less than about 80% carbon negative or carbon neutral components, such as less than about 60% carbon negative or carbon neutral components, such as less than about 40% carbon negative or carbon neutral components.

In order to produce high density polyethylene polymers in accordance with the present disclosure, a bio-based feedstock is collected, optionally converted, and purified to form a monomer, particularly a bio-based ethylene monomer, that is carbon negative or at least carbon neutral according to the mass balance approach described above. Bio-based ethylene can be formed in various different ways from various different feedstocks. The following processes for producing bio-based ethylene are exemplary and are believed capable of producing ethylene at purity levels necessary for many end-use applications, including using the resulting high density polyethylene polymer in a biomedical application.

In one embodiment, a biogas is collected and/or produced from a biomass resource and converted into ethylene. In one aspect, the biogas is methane produced from solid waste landfills and anaerobic digestion plants. Alternatively, the methane can be collected as a recycled gas from an industrial process. For example, methane is commonly released or incinerated into the environment instead of being collected and reused. By collecting a byproduct gas from an industrial process, the carbon footprint of the resulting monomer is greatly reduced.

Using a biogas as a starting feedstock for producing an ethylene monomer may provide various advantages and benefits depending upon the particular application. For example, biogases can contain little impurities which also prevents impurities from showing up in the final product.

Conversion of a methane biogas to ethanol can be carried out using different processes and steps. In one embodiment, for instance, methane can be directly converted into ethanol via the partial oxidation of methane in the presence of a metal-containing zeolite catalyst. In this embodiment, two mols of methane is reacted with 0.5 mols of molecular oxygen to yield ethanol.

In an alternative embodiment, the biogas methane can be converted into a syngas, which is produced by steam reforming the methane. The syngas, for instance, can contain carbon monoxide or carbon dioxide. Ethanol can then be produced from the carbon monoxide or the carbon dioxide.

The ethylene monomer can then be produced from ethanol. There are various different processes and techniques for converting ethanol to ethylene. In one embodiment, ethanol can be dehydrated in order to form ethylene. For example, in one embodiment, the resulting ethanol product can be optionally filtered and fed to a concentrator which may comprise one or more distillation columns. The distillation column can produce an ethanol rich stream that can then be converted to ethylene. For instance, the ethanol rich stream can be fed to a dehydrator. Dehydration can be conducted at an elevated temperature to produce water and ethylene together. As the product is cooled, water blended with the ethylene can be condensed and removed. The ethylene can then be condensed into liquid form if desired. The condensed ethylene can also be fed to a distillation column for further purification.

In an alternative embodiment, the biogas, such as methane, can be converted into ethylene without first being converted into ethanol. For example, in one embodiment, bio-based methane can first be converted into acetylene. The acetylene can then be converted into ethylene through a non-catalytic hydrogenation reaction.

The methane, for instance, can be converted into acetylene by a pyrolysis or a partial oxidation process. For instance, methane can be preheated and combined with oxygen at sub-stoichiometric amounts at temperatures of from about 500° C. to about 800° C. The mixture can be fed to a pyrolysis zone at a temperature of greater than about 1400° C., such as greater than about 1500° C. Acetylene is then produced and cooled by partial quenching. The acetylene at a temperature of from about 750° C. to about 950° C. is then hydrogenated to produce ethylene, optionally in the presence of ethane, which can also be bio-based.

In still another embodiment, ethanol is produced from a carbonaceous feedstock, such as a biomass. In one aspect, for instance, biomass can be fed to a fermentation process to produce ethanol from microorganisms. The biomass, for instance, can be any suitable plant matter, such as sugar cane. The biomass can also be any suitable cellulosic material or byproduct.

In one embodiment, a carbonaceous feedstock is first reformed to produce carbon dioxide, carbon monoxide, and/or hydrogen. The resulting gas stream can then be subjected to bacterial fermentation to produce ethanol. Microorganisms that can be used to produce ethanol include anaerobic bacteria. The anaerobic bacteria can be from the clostridium species, such as C. tjungdahffi, C. carboxydivorans, C. ragsdalei, and/or C. autoethanogenum.

Once ethanol is produced, the ethanol can be converted into ethylene as described above using a dehydration step.

In still another embodiment, biomass can be fermented directly to produce ethanol. For instance, cellulose, sugars and starches can be directly converted into ethanol using a fermentation process.

In still another embodiment, oils derived from biomass can be converted into ethylene. For example, vegetable oil or animal fat can be subjected to a hydrodeoxygenation process. More particularly, vegetable oil and/or animal fat can be hydrodeoxygenated in a manner that converts triglyceride and other molecules into paraffinic hydrocarbons, particularly ethylene. The ethylene can be purified, such as through filtration and distillation, and then used to produce the high density polyethylene polymers of the present disclosure.

In still another embodiment, gaseous ethylene can be produced form bio-based feedstocks using microorganisms, such as genetically engineered microorganisms. Metabolic pathways that can be used to produce gaseous ethylene include S-adenosyl-methionine pathway, 4-(methylsulfanyl)-2-oxobutanoate pathway and/or 2-oxoglutarate pathway. Directly producing ethylene gas, in some embodiments, can not only facilitate later polymer production but can also facilitate reduction of impurities.

In yet another embodiment, tall oil can be collected from a biomass feedstock and converted into ethylene. For example, in one embodiment, tall oil can be derived from a cellulosic feedstock.

Once the bio-based monomer is synthesized and purified, a high density polyethylene polymer is produced from the monomer. The high density polyethylene polymer can be produced exclusively from the bio-based monomer. In an alternative embodiment, however, the high density polyethylene polymer can be produced from a mixture of monomers including a bio-based monomer combined with a fossil-based ethylene monomer. For example, when a fossil-based ethylene monomer is used, the weight ratio between the bio-based monomer and the fossil-based monomer can be from about 1:95 to about 95:1.

The high density polyethylene can have a density of about 0.92 g/cm³ or greater, such as about 0.94 g/cm³ or greater, such as about 0.95 g/cm³ or greater, and generally less than about 1 g/cm³.

The high density polyethylene can be a high molecular weight polyethylene, a very high molecular weight polyethylene, and/or an ultrahigh molecular weight polyethylene. “High molecular weight polyethylene” refers to polyethylene compositions with an average molecular weight of at least about 2×10⁵ g/mol and, as used herein, is intended to include very-high molecular weight polyethylene and ultra-high molecular weight polyethylene. For purposes of the present specification, the molecular weights referenced herein are determined in accordance with the Margolies equation (“Margolies molecular weight”).

“Very-high molecular weight polyethylene” refers to polyethylene compositions with a molecular weight of from about 1×10⁶ g/mol and to about 3×10⁶ g/mol.

“Ultra-high molecular weight polyethylene” refers to polyethylene compositions with an average molecular weight of at least about 3×10⁶ g/mol and can be defined by ASTM D4020 or ISO 11542-1. In some embodiments, the molecular weight of the ultra-high molecular weight polyethylene composition is between about 3×10⁶ g/mol and about 30×10⁶ g/mol, or between about 3×10⁶ g/mol and about 20×10⁶ g/mol, or between about 3×10⁶ g/mol and about 10×10⁶ g/mol, or between about 3×10⁶ g/mol and about 6×10⁶ g/mol.

In one aspect, the high density polyethylene is a homopolymer of ethylene. In another embodiment, the high density polyethylene may be a copolymer. For instance, the high density polyethylene may be a copolymer of ethylene and another olefin containing from 3 to 16 carbon atoms, such as from 3 to 10 carbon atoms, such as from 3 to 8 carbon atoms. These other olefins include, but are not limited to, propylene, 1-butene, 1-pentene, 1-hexene, 1-heptene, 1-octene, 4-methylpent-1-ene, 1-decene, 1-dodecene, 1-hexadecene and the like. Also utilizable herein are polyene comonomers such as 1,3-hexadiene, 1,4-hexadiene, cyclopentadiene, dicyclopentadiene, 4-vinylcyclohex-1-ene, 1,5-cyclooctadiene, 5-vinylidene-2-norbornene and 5-vinyl-2-norbornene. However, when present, the amount of the non-ethylene monomer(s) in the copolymer may be less than about 10 mol. %, such as less than about 5 mol. %, such as less than about 2.5 mol. %, such as less than about 1 mol. %, wherein the mol. % is based on the total moles of monomer in the polymer. In accordance with the present disclosure, the comonomer can be a bio-based comonomer.

In one embodiment, the high density polyethylene may have a monomodal molecular weight distribution. Alternatively, the high density polyethylene may exhibit a bimodal molecular weight distribution. For instance, a bimodal distribution generally refers to a polymer having a distinct higher molecular weight and a distinct lower molecular weight (e.g. two distinct peaks) on a size exclusion chromatography or gel permeation chromatography curve. In another embodiment, the high density polyethylene may exhibit more than two molecular weight distribution peaks such that the polyethylene exhibits a multimodal (e.g., trimodal, tetramodal, etc.) distribution. Alternatively, the high density polyethylene may exhibit a broad molecular weight distribution wherein the polyethylene is comprised of a blend of higher and lower molecular weight components such that the size exclusion chromatography or gel permeation chromatography curve does not exhibit at least two distinct peaks but instead exhibits one distinct peak broader than the individual component peaks.

Any method known in the art can be utilized to synthesize the polyethylene. The polyethylene powder is typically produced by the catalytic polymerization of ethylene monomer or optionally with one or more other 1-olefin co-monomers, the 1-olefin content in the final polymer being less or equal to 10% of the ethylene content, with a heterogeneous catalyst and an organo aluminum or magnesium compound as cocatalyst. The ethylene is usually polymerized in gaseous phase or slurry phase at relatively low temperatures and pressures. The polymerization reaction may be carried out at a temperature of between 50° C. and 100° C. and pressures in the range of 0.02 and 2 MPa.

The molecular weight of the polyethylene can be adjusted by adding hydrogen. Altering the temperature and/or the type and concentration of the co-catalyst may also be used to fine tune the molecular weight. Additionally, the reaction may occur in the presence of antistatic agents to avoid fouling and product contamination.

Suitable catalyst systems include but are not limited to Ziegler-Natta type catalysts. Typically Ziegler-Natta type catalysts are derived by a combination of transition metal compounds of Groups 4 to 8 of the Periodic Table and alkyl or hydride derivatives of metals from Groups 1 to 3 of the Periodic Table. Transition metal derivatives used usually comprise the metal halides or esters or combinations thereof. Exemplary Ziegler-Natta catalysts include those based on the reaction products of organo aluminum or magnesium compounds, such as for example but not limited to aluminum or magnesium alkyls and titanium, vanadium or chromium halides or esters. The heterogeneous catalyst might be either unsupported or supported on porous fine grained materials, such as silica or magnesium chloride. Such support can be added during synthesis of the catalyst or may be obtained as a chemical reaction product of the catalyst synthesis itself.

In one embodiment, a suitable catalyst system can be obtained by the reaction of a titanium(IV) compound with a trialkyl aluminum compound in an inert organic solvent at temperatures in the range of −40° C. to 100° C., preferably −20° C. to 50° C. The concentrations of the starting materials are in the range of 0.1 to 9 mol/L, preferably 0.2 to 5 mol/L, for the titanium(IV) compound and in the range of 0.01 to 1 mol/L, preferably 0.02 to 0.2 mol/L for the trialkyl aluminum compound. The titanium component is added to the aluminum component over a period of 0.1 min to 60 min, preferably 1 min to 30 min, the molar ratio of titanium and aluminum in the final mixture being in the range of 1:0.01 to 1:4.

In another embodiment, a suitable catalyst system is obtained by a one or two-step reaction of a titanium(IV) compound with a trialkyl aluminum compound in an inert organic solvent at temperatures in the range of −40° C. to 200° C., preferably −20° C. to 150° C. In the first step the titanium(IV) compound is reacted with the trialkyl aluminum compound at temperatures in the range of −40° C. to 100° C., preferably −20° C. to 50° C. using a molar ratio of titanium to aluminum in the range of 1:0.1 to 1:0.8. The concentrations of the starting materials are in the range of 0.1 to 9.1 mol/L, preferably 5 to 9.1 mol/L, for the titanium(IV) compound and in the range of 0.05 and 1 mol/L, preferably 0.1 to 0.9 mol/L for the trialkyl aluminum compound. The titanium component is added to the aluminum compound over a period of 0.1 min to 800 min, preferably 30 min to 600 min. In a second step, if applied, the reaction product obtained in the first step is treated with a trialkyl aluminum compound at temperatures in the range of -10° C. to 150° C., preferably 10° C. to 130° C. using a molar ratio of titanium to aluminum in the range of 1:0.01 to 1:5.

In yet another embodiment, a suitable catalyst system is obtained by a procedure wherein, in a first reaction stage, a magnesium alcoholate is reacted with a titanium chloride in an inert hydrocarbon at a temperature of 50° to 100° C. In a second reaction stage the reaction mixture formed is subjected to heat treatment for a period of about 10 to 100 hours at a temperature of 110° to 200° C. accompanied by evolution of alkyl chloride until no further alkyl chloride is evolved, and the solid is then freed from soluble reaction products by washing several times with a hydrocarbon.

Each of the above-mentioned catalysts may further comprise an internal electron donor. Such donors may be selected from the group of linear and cyclic ethers; esters and diesters, such as aromatic esters; nitrogen-containing compounds; and sulphur containing compounds, such as thioethers. In one embodiment, the internal electron donor can be a derivative of succinic acid. In an alternative embodiment, the internal electron donor can be a substituted phenylene diester.

The Ziegler-Natta catalyst is used together with an activator. Suitable activators are metal alkyl compounds and especially aluminium alkyl compounds. These compounds include alkyl aluminium halides, such as ethylaluminium dichloride, diethylaluminium chloride, ethylaluminium sesquichloride, dimethylaluminium chloride and the like. They also include trialkylaluminium compounds, such as trimethylaluminium, triethylaluminium, tri-isobutylaluminium, trihexylaluminium and tri-n-octylaluminium. Furthermore they include alkylaluminium oxy-compounds, such as methylaluminiumoxane (MAO), hexaisobutylaluminiumoxane (HIBAO) and tetraisobutylaluminiumoxane (TIBAO). Also other aluminium alkyl compounds, such as isoprenylaluminium, may be used. Especially preferred activators are trialkylaluminiums, of which triethylaluminium, trimethylaluminium and tri-isobutylaluminium are particularly used.

The amount in which the activator is used depends on the specific catalyst and activator. Typically triethylaluminium is used in such amount that the molar ratio of aluminium to the transition metal, like Al/Ti, is from 1 to 1000, preferably from 3 to 100 and in particular from about 5 to about 30 mol/mol.

It is possible to use external donors with the catalyst. The use of such donors is known in the art. They may be selected from linear and cyclic ethers, esters, silicon ethers, nitrogen-containing compounds and such.

Utilizing a catalyst system as described above, the high density polyethylene polymer can be produced in a slurry polymerization process. For instance, the catalyst can be introduced into a slurry containing ethylene and a diluent.

The slurry polymerization step for producing the ultra-high molecular weight polyethylene is conducted at a temperature of from 30 to 110 C°. Preferably, the temperature is from 35 to 75° C. and more preferably from 40 to 70° C., such as from 42 to 70° C. or from 45 to 70° C. The molecular weight of the polymer produced in the process tends to be higher when operating at the lower end of the temperature range. On the other hand, the polymerization rate tends to increase with increasing temperature. The above-described ranges offer a good compromise between the molecular weight capability and the productivity.

The pressure in the slurry polymerization step for producing the ultra-high molecular weight polyethylene is not really critical and may be chosen freely within a range of from about 1 to about 100 bar (absolute pressure). The choice of the operating pressure depends, among others, on the choice of the diluent used in the polymerization.

The diluent in the slurry polymerization step for producing the ultra-high molecular weight polyethylene may be any suitable diluent which dissolves ethylene but not the high density polyethylene in the reaction conditions. Furthermore the diluent should not react with the polymerization catalyst. Preferably the diluent is selected from alkanes having from 2 to 8 carbon atoms and their mixtures. More preferably the diluent is selected from the group consisting of propane, isobutane, n-butane and mixtures thereof.

The slurry polymerization for producing the ultra-high molecular weight polyethylene may be conducted batch-wise or continuously.

The ethylene content in the fluid phase of the slurry may be from 1 to about 50% by mole, preferably from about 2 to about 20% by mole and in particular from about 2 to about 10% by mole. The benefit of having a high ethylene concentration is that the productivity of the catalyst is increased but the drawback is that more ethylene then needs to be recycled than if the concentration was lower.

The slurry polymerization for producing the ultra-high molecular weight polyethylene may be conducted in any known reactor used for slurry polymerization. Such reactors include a continuous stirred tank reactor and a loop reactor. It is especially preferred to conduct the polymerization in a loop reactor. such reactors the slurry is circulated with a high velocity along a closed pipe by using a circulation pump.

The average residence time in the slurry polymerization step is typically from 20 to 120 minutes, preferably from 30 to 80 minutes. As it is well known in the art the average residence time T for a continuous process can be calculated from:

$$τ = \frac{{V}_{R}}{{Q}_{o}}$$

where V_{R} is the volume of the reaction space (in case of a loop reactor, the volume of the reactor) and Qₒ is the volumetric flow rate of the product stream (including the polymer product and the fluid reaction mixture).

The high density polyethylene polymer generally has a molecular weight of greater than about 200,000 g/mol, such as greater than about 300,000 g/mol. For instance, the polyethylene polymer can have an average molecular weight of greater than about 500,000 g/mol, such as greater than about 700,000 g/mol, such as greater than about 1,000,000 g/mol, such as greater than about 1,300,000 g/mol, such as greater than about 1,700,000 g/mol, such as greater than about 2,000,000 g/mol, such as greater than about 2,500,000 g/mol, such as greater than about 3,000,000 g/mol, such as greater than about 3,500,000 g/mol, such as greater than about 4,000,000 g/mol, such as greater than about 4,500,000 g/mol, such as greater than about 5,000,000 g/mol, such as greater than about 5,500,000 g/mol, such as greater than about 6,000,000 g/mol, such as greater than about 6,500,000 g/mol, such as greater than about 7,000,000 g/mol, such as greater than about 7,500,000 g/mol, such as greater than about 8,000,000 g/mol, and less than about 12,000,000 g/mol.

The polyethylene polymer can have a melt flow rate of from about 0.1 g/10 min to about 50 g/10 min. The melt flow rate of the polymer is determined according to ASTM Test D1238 @ 190° C. and at a load of 21.5 kg. In one embodiment, the high density polyethylene polymer has a relatively low melt flow rate, such as less than about 30 g/10 min, such as less than about 20 g/10 min, such as less than about 10 g/10 min, such as less than about 5 g/10 min, such as less than about 4 g/10 min, such as less than about 3 g/10 min, such as less than about 2 g/10 min, such as less than about 1 g/10 min. In one embodiment, the melt flow rate is so low that it cannot be measured according to the ASTM test described above.

In addition to the mass balance approach, the high density polyethylene polymer produced according to the present disclosure can then be measured for bio-based content using ASTM Test D6866 (2021). The above analytical test was developed in order to determine the bio-based content of solid, liquid or gaseous samples using radiocarbon dating. ASTM Test D6866 distinguishes carbon resulting from contemporary biomass-based inputs from those derived from fossil-based inputs. More particularly, the method relies on determining the amount of radiocarbon dating isotope ¹⁴C (half-life of 5,730 years) in the polymer. The method identifies whether the carbon contained in the polymer derives from a bio source, such as modern plant or animals, or from a fossil source, or from a mixture of these. Carbon from fossil sources generally has a ¹⁴C amount very close to zero. Measuring the ¹⁴C isotope amount of the high density polyethylene polymer can verify that all or a portion of the material or article derives from a bio-source. ASTM Test D6866 includes methods A-C. In one embodiment, method B may be used.

High density polyethylene polymers made according to the present disclosure, when tested according to ASTM Test D6866, can have a bio-based content of at least 10% based on radiocarbon dating of Total Organic Carbon Content. For example, the high density polyethylene polymer can have a bio-based content of greater than about 20%, such as greater than about 30%, such as greater than about 40%, such as greater than about 50%, such as greater than about 60%, such as greater than about 70%, such as greater than about 80%. In one embodiment, the high density polyethylene polymer can be made exclusively from a bio-based feedstock and have a 100% bio-based content. In other embodiments, the high density polyethylene polymer can be made partially from fossil-based ethylene such that the bio-based content is less than about 90%, such as less than about 80%, such as less than about 70%, such as less than about 60%, such as less than about 50%, such as less than about 40%, such as less than about 30%.

The high density polyethylene polymer produced according to the present disclosure is generally collected in the form of particles for use in making various different products and articles.

In one embodiment, the polyethylene particles are made from a polyethylene polymer having a relatively low bulk density as measured according to DIN53466. For instance, in one embodiment, the bulk density is generally less than about 0.4 g/cm³, such as less than about 0.35 g/cm³, such as less than about 0.33 g/cm³, such as less than about 0.3 g/cm³, such as less than about 0.28 g/cm³, such as less than about 0.26 g/cm³. The bulk density is generally greater than about 0.1 g/cm³, such as greater than about 0.15 g/cm³. In one embodiment, the polymer has a bulk density of from about 0.2 g/cm³ to about 0.27 g/cm³.

In one embodiment, the polyethylene particles can be a free-flowing powder. The particles can have a median particle size (d50) by volume of less than 250 microns. For example, the median particle size (d50) of the polyethylene particles can be less than about 150 microns, such as less than about 125 microns. The median particle size (d50) is generally greater than about 10 microns. The powder particle size can be measured utilizing a laser diffraction method according to ISO 13320.

In one embodiment, 90% of the polyethylene particles can have a particle size of less than about 250 microns. In other embodiments, 90% of the polyethylene particles can have a particle size of less than about 200 microns, such as less than about 170 microns.

The polyethylene may have a viscosity number of from at least 100 mL/g, such as at least 500 mL/g, such as at least 1,500 mL/g, such as at least 2,000 mL/g, such as at least 4,000 mL/g to less than about 6,000 mL/g, such as less than about 5,000 mL/g, such as less than about 4000 mL/g, such as less than about 3,000 mL/g, such as less than about 1,000 mL/g, as determined according to ISO 1628 part 3 utilizing a concentration in decahydronapthalene of 0.0002 g/mL.

The high density polyethylene may have a crystallinity of from at least about 40% to 85%, such as from 45% to 80%.

In producing products and articles, the high density polyethylene polymer can be combined with various additives, such as heat stabilizers, light stabilizers, UV absorbers, acid scavengers, flame retardants, lubricants, colorants, and the like.

In one embodiment, a heat stabilizer may be present in the composition. The heat stabilizer may include, but is not limited to, phosphites, aminic antioxidants, phenolic antioxidants, or any combination thereof.

In one embodiment, an antioxidant may be present in the composition. The antioxidant may include, but is not limited to, secondary aromatic amines, benzofuranones, sterically hindered phenols, or any combination thereof.

In one embodiment, a light stabilizer may be present in the composition. The light stabilizer may include, but is not limited to, 2-(2′-hydroxyphenyl)-benzotriazoles, 2-hydroxy-4-alkoxybenzophenones, nickel containing light stabilizers, 3,5-di-tert-butyl-4-hydroxbenzoates, sterically hindered amines (HALS), or any combination thereof.

In one embodiment, a UV absorber may be present in the composition in lieu of or in addition to the light stabilizer. The UV absorber may include, but is not limited to, a benzotriazole, a benzoate, or a combination thereof, or any combination thereof.

In one embodiment, a halogenated flame retardant may be present in the composition. The halogenated flame retardant may include, but is not limited to, tetrabromobisphenol A (TBBA), tetrabromophthalic acid anhydride, dedecachloropentacyclooctadecadiene (dechlorane), hexabromocyclodedecane, chlorinated paraffins, or any combination thereof.

In one embodiment, a non-halogenated flame retardant may be present in the composition. The non-halogenated flame retardant may include, but is not limited to, resorcinol diphosphoric acid tetraphenyl ester (RDP), ammonium polyphosphate (APP), phosphine acid derivatives, friaryl phosphates, trichloropropylphosphate (TCPP), magnesium hydroxide, aluminum trihydroxide, antimony trioxide.

In one embodiment, a lubricant may be present in the composition. The lubricant may include, but is not limited to, silicone oil, waxes, molybdenum disulfide, or any combination thereof.

In one embodiment, a colorant may be present in the composition. The colorant may include, but is not limited to, inorganic and organic based color pigments.

In one aspect, an acid scavenger may be present in the polymer composition. The acid scavenger, for instance, may comprise an alkali metal salt or an alkaline earth metal salt. The salt can comprise a salt of a fatty acid, such as a stearate. Other acid scavengers include carbonates, oxides, or hydroxides. Particular acid scavengers that may be incorporated into the polymer composition include a metal stearate, such as calcium stearate. Still other acid scavengers include zinc oxide, calcium carbonate, magnesium oxide, and mixtures thereof.

These additives may be used singly or in any combination thereof. In general, each additive may be present in the polymer composition or in the resulting polymer article in an amount of at least about 0.05 wt. %, such as in an amount of at least about 0.1 wt. %, such as at least about 0.25 wt. %, such as at least about 0.5 wt. %, such as at least about 1 wt. % and generally less than about 20 wt. %, such as less than about 10 wt. %, such as less than about 5 wt. %, such as less than about 4 wt. %, such as less than about 2 wt. %. The sum of the wt. % of all of the components, including any additives if present, utilized in the polymer composition and articles will be 100 wt. %.

The high density polyethylene polymer made in accordance with the present disclosure can be used in numerous and diverse applications to produce all different types of products and articles. The manner in which the high density polyethylene polymer is formed into various articles can also vary. In one embodiment, for instance, the high density polyethylene particles can be combined with a plasticizer and fed through a gel extrusion process for producing articles, such as fibers and films. During gel extrusion, significant amounts of a plasticizer are combined with the high density polyethylene polymer in order to form a gel that can be extruded through a die. Once a polymer article is formed, the plasticizer is then removed from the final product.

When forming gel extruded articles, the high density polyethylene polymer is combined with the plasticizer to form a polymer composition.

In general, the high density polyethylene particles are present in the polymer composition in an amount up to about 50% by weight. For instance, the high density polyethylene particles can be present in the polymer composition in an amount less than about 45% by weight, such as in an amount less than about 40% by weight, such as in an amount less than about 35% by weight, such as in an amount less than about 30% by weight, such as in an amount less than about 25% by weight, such as in an amount less than about 20% by weight, such as in an amount less than about 15% by weight. The polyethylene particles can be present in the composition in an amount greater than about 5% by weight, such as in an amount greater than about 10% by weight, such as in an amount greater than about 15% by weight, such as in an amount greater than about 20% by weight, such as in an amount greater than about 25% by weight. During gel processing, a plasticizer is combined with the high density polyethylene particles which can be substantially or completely removed in forming polymer articles. For example, in one embodiment, the resulting polymer article can contain the high density polyethylene polymer in an amount greater than about 70% by weight, such as in an amount greater than about 80% by weight, such as in an amount greater than about 85% by weight, such as in an amount greater than about 90% by weight, such as in an amount greater than about 95% by weight.

In general, any suitable plasticizer can be used during the gel extruding process. The plasticizer, for instance, may comprise a hydrocarbon oil, an alcohol, an ether, an ester such as a diester, or mixtures thereof. For instance, suitable plasticizers include mineral oil, a paraffinic oil, decaline, and the like. Other plasticizers include xylene, dioctyl phthalate, dibutyl phthalate, stearyl alcohol, oleyl alcohol, decyl alcohol, nonyl alcohol, diphenyl ether, n-decane, n-dodecane, octane, nonane, kerosene, toluene, naphthalene, tetraline, and the like. In one embodiment, the plasticizer may comprise a halogenated hydrocarbon, such as monochlorobenzene. Cycloalkanes and cycloalkenes may also be used, such as camphene, methane, dipentene, methylcyclopentandiene, tricyclodecane, 1,2,4,5-tetramethyl-1,4-cyclohexadiene, and the like. The plasticizer may comprise mixtures and combinations of any of the above as well.

The plasticizer is generally present in the composition used to form the polymer articles in an amount greater than about 50% by weight, such as in an amount greater than about 55% by weight, such as in an amount greater than about 60% by weight, such as in an amount greater than about 65% by weight, such as in an amount greater than about 70% by weight, such as in an amount greater than about 75% by weight, such as in an amount greater than about 80% by weight, such as in an amount greater than about 85% by weight, such as in an amount greater than about 90% by weight, such as in an amount greater than about 95% by weight, such as in an amount greater than about 98% by weight. In fact, the plasticizer can be present in an amount up to about 99.5% by weight.

The high density polyethylene particles and plasticizer to form a homogeneous gel-like material. In order to form polymer articles in accordance with the present disclosure, the high density polyethylene particles are combined with the plasticizer and extruded through a die of a desired shape. In one embodiment, the composition can be heated within the extruder. For example, the plasticizer can be combined with the particle mixture and fed into an extruder. In accordance with the present disclosure, the plasticizer and particle mixture form a homogeneous gel-like material prior to leaving the extruder for forming polymer articles with little to no impurities.

In one embodiment, elongated articles are formed during the gel spinning or extruding process. The polymer article, for instance, may be in the form of a fiber or a film, such as a membrane.

During the process, at least a portion of the plasticizer is removed from the final product. The plasticizer removal process may occur due to evaporation when a relatively volatile plasticizer is used. Otherwise, an extraction liquid can be used to remove the plasticizer. The extraction liquid may comprise, for instance, a hydrocarbon solvent. One example of the extraction liquid, for instance, is dichloromethane. Other extraction liquids include acetone, chloroform, an alkane, hexene, heptene, an alcohol, or mixtures thereof.

If desired, the resulting polymer article can be stretched at an elevated temperature below the melting point of the polymer mixture to increase strength and modulus. Suitable temperatures for stretching are in the range of from about ambient temperature to about 155° C. The draw ratios can generally be greater than about 4, such as greater than about 6, such as greater than about 8, such as greater than about 10, such as greater than about 15, such as greater than about 20, such as greater than about 25, such as greater than about 30. In certain embodiments, the draw ratio can be greater than about 50, such as greater than about 100, such as greater than about 110, such as greater than about 120, such as greater than about 130, such as greater than about 140, such as greater than about 150. Draw ratios are generally less than about 1,000, such as less than about 800, such as less than about 600, such as less than about 400. In one embodiment, lower draw ratios are used such as from about 4 to about 10. The polymer article can be uniaxially stretched or biaxially stretched.

Polymer articles made in accordance with the present disclosure have numerous uses and applications. For example, in one embodiment, the process is used to produce a membrane. The membrane can be used, for instance, as a battery separator. Alternatively, the membrane can be used as a microfilter. When producing fibers, the fibers can be used to produce nonwoven fabrics, ropes, nets, and the like. In one embodiment, the fibers can be used as a filler material in ballistic apparel.

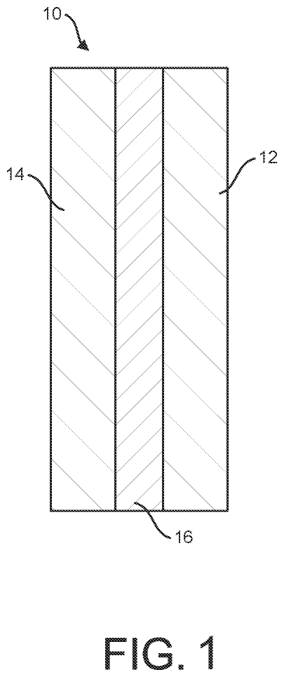

Referring to FIG. 1, one embodiment of a lithium ion battery 10 made in accordance with the present disclosure is shown. The battery 10 includes an anode 12 and a cathode 14. The anode 12, for instance, can be made from a lithium metal. The cathode 14, on the other hand, can be made from sulfur or from an intercalated lithium metal oxide. In accordance with the present disclosure, the battery 10 further includes a porous membrane 16 or separator that is positioned between the anode 12 and the cathode 14. The porous membrane 16 minimizes electrical shorts between the two electrodes while allowing the passage of ions, such as lithium ions. As shown in FIG. 1, in one embodiment, the porous membrane 16 is a single layer polymer membrane and does not include a multilayer structure. In one aspect, the single layer polymer membrane may also include a coating. The coating can be an inorganic coating made from, for instance, aluminum oxide or a titanium oxide. Alternatively, the single layer polymer membrane may also include a polymeric coating. The coating can provide increased thermal resistance.

In an alternative embodiment, the high density polyethylene polymer can be used to produce various different biomaterials, such as implants. For instance, since the high density polyethylene polymer can be biocompatible, the polymer is well suited to producing prosthetic knee joints, prosthetic hip joints, and other prosthetic replacement joints for the human or animal body. For example, in one embodiment, the high density polyethylene can be used as the lining of an acetabular cup of a prosthetic hip joint.

When used as a biomaterial, the high density polyethylene polymer should have little to no impurities. In this regard, the high density polyethylene polymer can have an ash content of less than about 500 ppm, such as less than about 250 ppm, such as less than about 100 ppm, such as less than about 50 ppm, such as less than about 10 ppm. In fact, in certain embodiments, the ash content can be less than about 8 ppm, such as less than about 5 ppm, such as less than about 2 ppm. As used herein, ash content is determined according to ASTM Test D5630-13.

The high density polyethylene polymer can be used to produce all different types of biomedical products including all different types of implants. The implant can be designed for the human body or for an animal body, including all vertebrates. The polymer, for instance, can be used to produce implants for dogs, cats, sheep, horses, cows, and the like.

In one embodiment, sintered products can be made from the high density polyethylene polymer, particularly porous articles. Porous articles may be formed by a free sintering process which involves introducing the polyethylene polymer powder described above into either a partially or totally confined space, e.g., a mold, and subjecting the molding powder to heat sufficient to cause the polyethylene particles to soften, expand and contact one another. Suitable processes include compression molding and casting. The mold can be made of steel, aluminum or other metals. The polyethylene polymer powder used in the molding process is generally ex-reactor grade, by which is meant the powder does not undergo sieving or grinding before being introduced into the mold. The additives discussed above may of course be mixed with the powder.

The mold is heated in a convection oven, hydraulic press or infrared heater to a sintering temperature between about 140° C. and about 300° C., such as between about 160° C. and about 300° C., for example between about 170° C. and about 240° C. to sinter the polymer particles. The heating time and temperature vary and depend upon the mass of the mold and the geometry of the molded article. However, the heating time typically lies within the range of about 25 to about 100 minutes. During sintering, the surface of individual polymer particles fuse at their contact points forming a porous structure. Subsequently, the mold is cooled and the porous article removed. In general, a molding pressure is not required. However, in cases requiring porosity adjustment, a proportional low pressure can be applied to the powder.

Porous substrates made in accordance with the present disclosure have been found to have an excellent blend of properties. For instance, porous substrates made in accordance with the present disclosure can have a relatively low pressure drop, indicating excellent filter properties, in combination with a relatively high level of flexural strength, indicating a product that is less brittle and more flexibility. For instance, porous substrates made according to the present disclosure can have a pressure drop of less than 10 mbar, such as less than about 8 mbar, such as less than about 6 mbar, such as even less than about 4 mbar. In one embodiment, for instance, the pressure drop can be from about 0.1 mbar to about 3.5 mbar.

In addition, the porous substrate can have relatively high flexural strength. Flexural strength, for instance, can be determined in accordance with DIN ISO 178. The flexural strength of porous substrates made according to the present disclosure can generally be greater than about 1.5 MPa, such as greater than about 2 MPa, such as greater than about 2.2 MPa, such as greater than about 2.4 MPa, such as greater than about 2.6 MPa, such as greater than about 2.8 MPa, such as greater than about 3 MPa. The flexural strength is generally less than about 8 MPa.

In addition to the above properties, porous substrates made according to the present disclosure can have various other beneficial physical properties. For instance, the porous substrates can have a porosity of greater than about 30%, such as greater than about 35%, such as greater than about 40%. The porosity is generally less than about 80%, such as less than about 60%, such as less than about 55%. Porosity can be determined according to DIN Test 66133. Average pore size which can also be determined according to Test DIN 66133 can generally be greater than about 80 microns, such as greater than about 85 microns, such as greater than about 90 microns, such as greater than about 95 microns, such as greater than about 100 microns, such as greater than about 105 microns, such as greater than about 110 microns, such as greater than about 115 microns, such as greater than about 120 microns, such as even greater than about 125 microns. The average pore size is generally less than about 180 microns.

Porous substrates made according to the present disclosure can be used in numerous and diverse applications. Specific examples include wastewater aeration, capillary applications and filtration.

Aeration is the process of breaking down wastewater using microorganisms and vigorous agitation. The microorganisms function by coming into close contact with the dissolved and suspended organic matter. Aeration is achieved in practice by the use of “aerators” or “porous diffusers”. Aerators are made from many different materials and come in a few widely accepted shapes and geometries. The three main types of materials currently used in the manufacture of aerators are ceramics (including aluminum oxide, aluminum silicates and silica), membranes (mostly elastomers like ethylene/propylene dimers-EPDM and plastics (mostly HDPE).

The present porous articles provide attractive replacements for ceramic, membrane and HDPE aerators due to the fact the tighter control on particle size distribution and bulk density leads to the production of aerators with tightly controlled pores, consistent flow rates, larger bubble sizes and lower pressure drops. In addition, the incorporation UV stabilizer and/or antimicrobial additives should allow the performance of the present sintered porous polyethylene aerators to be further improved beyond that of existing aerators. Thus, the incorporation of UV stabilizers can be used to extend the life expectancy of the present aerators in outdoor environments, whereas the addition of antimicrobial agents should prevent fouling on the aerator surface, thereby allowing the aerators to perform at peak efficiency for longer periods.

Capillary applications of the present porous sintered articles include writing instruments, such as highlighters, color sketch pens, permanent markers and erasable whiteboard markers. These make use of the capillary action of a porous nib to transport ink from a reservoir to a writing surface. Currently, porous nibs formed from ultra-high molecular weight polyethylene are frequently used for highlighters and color sketch pens, whereas permanent and whiteboard markers are generally produced from by polyester (polyethylene terephthalate), polyolefin hollow fibers and acrylic porous materials. The large pore size of the present sintered articles make them attractive for use in the capillary transport of the alcohol-based high-viscosity inks employed in permanent markers and white board markers.

With regard to filtration applications, the present porous sintered articles are useful in, for example, produced water (drilling injection water) filtration. Thus, in crude oil production, water is often injected into an on-shore reservoir to maintain pressure and hydraulically drive oil towards a producing well. The water being injected has to be filtered so that it does not prematurely plug the reservoir or equipment used for this purpose. In addition as oil fields mature, the generation of produced water increases. Porous tubes made from the present polyethylene powder are ideal filtration media for produced water filtration because they are oleophilic, they can form strong and stable filter elements which are back-washable, abrasion resistant, chemically resistant and have a long service life.

The present porous sintered articles also find utility in other filtration applications, where oil needs to be separated from water, such as filtration of turbine and boiler water for power plants, filtration of cooling water emulsions, de-oiling of wash water from car wash plants, process water filtration, clean-up of oil spills from seawater, separating glycols from natural gas and aviation fuel filters.

Another application of the present porous sintered articles is in irrigation, where filtration of incoming water is necessary to remove the tiny sand particles that can clog sprinkler systems and damage other irrigation devices including pumps. The traditional approach to this issue has been the use of stainless steel screens, complex disc filters, sand media filters and cartridge filters. One of the key requirements of these filters is pore size, which is normally required to range from 100 μ to 150 μ. Other considerations are high flow rate, low pressure drop, good chemical resistance, high filter strength and long service life. The properties of the present porous sintered articles make them particularly qualified for such use.

A further filtration application is to replace the sediment filters used as pre-filters to remove rust and large sediments in multi-stage drinking water applications where sintered polyethylene filters have shown extended life over the more expensive carbon blocks, reverse osmosis membranes and hollow fiber cartridges. Until now the required sintered part strength of such filters was achievable only by blending LDPE or HDPE together with UHMWPE powder. However, these blends suffer from a number of disadvantages in that the pore size of the sintered filter is reduced and existing UHMWPE powders are unable to produce filters with pore sizes greater than 20 μ and with adequate part strength. In contrast, the present polyethylene powder facilitates the design of sediment filters which exhibit adequate part strength at pore sizes >30 μ and which show superior pore size retention during use at high water velocities.

Other filtration applications of the present porous sintered articles include medical fluid filtration, such as filtration of blood outside the human body, filtration to remove solids in chemical and pharmaceutical manufacturing processes, and filtration of hydraulic fluids to remove solid contaminants.

In a further filtration embodiment, the present polyethylene powder can be used in the production of carbon block filters. Carbon block filters are produced from granular activated carbon particles blended with about 5 wt % to about 80 wt %, generally about 15 wt % to about 25 wt % of a thermoplastic binder. The blend is poured into a mold, normally in the shape of a hollow cylinder, and compressed so as to compact the blended material as much as possible. The material is then heated to a point where the binder either softens or melts to cause the carbon particles to adhere to one another. Carbon block filters are used in a wide variety of applications, including water filtration, for example, in refrigerators, air and gas filtration, such as, the removal of toxic organic contaminants from cigarette smoke, organic vapor masks and gravity flow filtration devices.

These and other modifications and variations to the present invention may be practiced by those of ordinary skill in the art, without departing from the spirit and scope of the present invention, which is more particularly set forth in the appended claims. In addition, it should be understood that aspects of the various embodiments may be interchanged both in whole or in part. Furthermore, those of ordinary skill in the art will appreciate that the foregoing description is by way of example only and is not intended to limit the invention so further described in such appended claims.

What is claimed:

1. A polymer composition comprising:
polymer particles comprising a high density polyethylene polymer, the high density polyethylene polymer having an average molecular weight of greater than about 200,000 g/mol, the high density polyethylene polymer having a density of greater than about 0.92 g/cm³ (ISO 1183), the high density polyethylene polymer being formed from an ethylene monomer, wherein at least a portion of the ethylene monomer comprises or is derived from a carbon negative or carbon neutral component.

2. A polymer composition as defined in claim 1, wherein the high density polyethylene has an average molecular weight of greater than about 500,000 g/mol and less than about 12,000,000 g/mol.

3. A polymer composition as defined in claim 1, wherein the ethylene monomer is formed from the carbon negative or carbon neutral component.

4. A polymer composition as defined in claim 3, wherein the carbon negative or carbon neutral component comprises methane and wherein the methane is subjected to a pyrolysis or a partial oxidation process for forming acetylene, and wherein the acetylene is hydrogenated into ethylene.

5. A polymer composition as defined in claim 3, wherein the carbon negative or carbon neutral component comprises ethanol that is converted into ethylene.

6. A polymer composition as defined in claim 3, wherein the carbon negative or carbon neutral component comprises a vegetable oil or an animal fat and wherein the vegetable oil or animal fat is converted to ethylene by hydrodeoxygenation.

7. A polymer composition as defined in claim 3, wherein the carbon negative or carbon neutral component comprises a tall oil and wherein the tall oil is converted to ethylene.

8. A polymer composition as defined in claim 1, wherein the polymer particles have an average particle size, D50, of from about 10 microns to about 1,000 microns.

9. A polymer composition as defined in claim 1, wherein the high density polyethylene polymer has been Ziegler-Natta catalyzed.

10. A polymer composition as defined in claim 1, wherein the high density polyethylene polymer has a bulk density of from about 0.2 g/cm³ to about 0.54 g/cm³.

11. A polymer composition as defined in claim 1, wherein the high density polyethylene polymer has an MFR of from about 0 g/10 min. to about 10 g/10 min.

12. A polymer composition as defined in claim 1, wherein the high density polyethylene polymer comprises a polyethylene copolymer of ethylene and at least one comonomer comprising hexene, butene, propylene, or mixtures thereof.

13. A polymer composition as defined in claim 1, wherein the high density polyethylene polymer has been crosslinked.

14. A polymer composition as defined in claim 1, wherein the high density polyethylene polymer is formed from a mixture of a fossil-based ethylene monomer and a bio-based ethylene monomer.

15. A medical implant formed from the polymer composition as defined in claim 1.

16. A battery separator formed from the polymer composition as defined in claim 1, the battery separator comprising a porous membrane.

17. A battery separator as defined in claim 16, wherein the porous membrane includes a coating, the coating comprising an inorganic coating or a polymer coating.

18. A battery comprising an anode, a cathode, and the battery separator defined in claim 16, the battery separator being positioned between the anode and the cathode.

19. A filter element formed from the polymer composition as defined in claim 1, wherein the filter element comprises a sintered product.

20. A polymer composition comprising:
polymer particles comprising a high density polyethylene polymer, the high density polyethylene polymer having an average molecular weight of greater than about 300,000 g/mol, the high density polyethylene polymer having a density of greater than about 0.93 g/cm³, the high density polyethylene polymer being formed from an ethylene monomer, wherein at least a portion of the ethylene monomer comprises a bio-based ethylene such that the high density polyethylene polymer, when tested according to ASTM Test D6866-21, has at least 10% bio-based content based on radiocarbon dating of Total Organic Carbon Content.

21. A polymer composition as defined in claim 20, wherein the high density polyethylene polymer is formed from a mixture of a fossil-based ethylene monomer and a bio-based ethylene monomer.
